(19) United States
(12) Patent Application Publication
(10) Pub. No.: US 2022/0287937 A1
(43) Pub. Date: Sep. 15, 2022

(54) SKIN SERUM AND PROCESS OF MAKING THE SAME

(71) Applicant: JPB LABS, INC., New York, NY (US)

(72) Inventor: Andrew Shemin, New York, NY (US)

(21) Appl. No.: 17/680,116

(22) Filed: Feb. 24, 2022

Related U.S. Application Data
(60) Provisional application No. 63/153,261, filed on Feb. 24, 2021.

(51) Int. Cl. A61K 8/362; A61K 31/194; A61K 9/00; A61K 45/06; A61K 47/44; A61K 47/12; A61Q 19/08; A61P 17/16

(52) U.S. Cl. CPC A61K 8/362; A61K 31/194; A61K 9/0014; A61K 45/06; A61K 47/44; A61K 47/12; A61Q 19/08; A61P 17/16

(57) ABSTRACT

An anti-aging serum to slow the aging process by providing fuel to skin cells through topical application of a precursor Krebs cycle intermediate and associated fuel to dampen the effects of aging brought on by cellular replication and metabolism related irregularities. The serum improves skin experiencing mild to moderate wrinkling and mild to moderate mottled hyperpigmentation helping to achieve a more youthful look.

### CROSS-REFERENCE TO RELATED APPLICATIONS

This application claims priority to U.S. Provisional Application Ser. No. 63/153,261, which was filed Feb. 24, 2021, the disclosure of which is hereby incorporated by reference in its entirety and for all purposes.

### FIELD

This present disclosure relates generally to pharmaceutical compositions for skin treatment and, more specifically, but not exclusively, to a skin serum having a Krebs cycle intermediate precursor.

### BACKGROUND

After realizing the potential metabolism enhancing and anti-aging properties of Krebs cycle intermediates and precursors when ingested, interest developed towards adopting these Krebs cycle intermediates and precursors for use in skin care.

While conventional studies identified the effectiveness of adding a Krebs cycle intermediate or its precursor to skin treatment formulations, early efforts at creating such skin treatments using diethyl malate resulted in skin irritation and allergic skin reactions whereas the use of diethylhexyl malate resulted in a pasty oil mixture that is unappealing to the modern discerning customer.

Accordingly, a need exists for an improved pharmaceutical composition to overcome the aforementioned deficiencies of conventional skin serums.

### BRIEF DESCRIPTION OF THE DRAWINGS

FIG. 1 is a graph illustrating improvements to skin redness observed during a four-week clinical case study using one exemplary embodiment of a topical skin composition.

FIG. 2 is a graph showing the improvements in the evenness of the overall skin tone over a four-week clinical case study using the exemplary embodiment of a topical skin composition of FIG. 1.

It should be noted that the figures are not drawn to scale and that elements of similar structures or functions are generally represented by like reference numerals for illustrative purposes throughout the figures. It also should be noted that the figures are only intended to facilitate the description of the preferred embodiments. The figures do not illustrate every aspect of the described embodiments and do not limit the scope of the present disclosure.

### DETAILED DESCRIPTION OF PREFERRED EMBODIMENTS

Exemplary embodiments of serums comprising mixtures of Krebs cycle intermediates combined with fuel substrates and antioxidants that are proven to improve the appearance of skin and rejuvenate skin cells are disclosed. The mixtures are selected from specific Krebs cycle intermediate precursors, fuel substrates, and antioxidants.

Krebs cycle intermediates are the acids or salts of compounds that are utilized during the Krebs tricarboxylic acid cycle. These compounds, depending on composition, yield different amounts of ATP. It is believed that a number of disorders involved altered oxidative metabolism, including skin botching, skin elasticity loss, and wrinkle formation, involve disruption of the Krebs cycle.

Precursors of Krebs cycle intermediates are compounds that, upon administration to a subject, are converted by cells into Krebs cycle intermediates. Generally, mono- and di-alkyl citrates, aconitates, isocitrates, α-ketoglutarates, succinates, fumarates, malates, maleates, and oxaloacetates are desirable precursors because their bonds are readily broken by cells to yield Krebs cycle intermediates.

Krebs cycle intermediates or precursors of Krebs cycle intermediates that are acidic tend to irritate the skin, especially at high concentrations. Thus, preferred embodiments would feature Krebs cycle intermediates or Krebs cycle intermediate precursors that are less acidic or irritating to the skin.

Preferred Krebs cycle intermediate precursors are a combination of diethylhexyl maleate and diauryl citrate which in combination with the formulation in the preferred embodiment, are less irritating to the skin.

A serum is disclosed that uses a combination of Krebs cycle intermediate precursors to help rejuvenate skin cells by providing skin cells with energy to adjust cell metabolism and facilitate skin cell rejuvenation. The serum also includes antioxidants derived from grapes, resveratrol, whose targeted application to the skin can help skin cells fight off oxidative damage. The serum uses oil and castor oil both as a fuel for the Krebs cycle intermediate precursors as well as a vehicle to hold the active ingredients in distilled water creating a serum that is both effect and feels nice when applied.

Furthermore, the serum features a high-water content (86% wt) overcoming the typical drawbacks of creams and lotions as being oily leading to a favorable feel and texture as reported by test subjects.

The serum formulation yields a decreased rates of skin irritation with statistically significant improvements to skin conditions when tested on a sample size of 31 test subjects over a 4-week trial period with only one report of skin irritation.

One preferred ingredient of the inventive skin serum is a Krebs cycle intermediate precursor. This precursor is known to be metabolized in the body to promote healthy mitochondrial metabolism.

In a preferred embodiment, the Krebs cycle intermediate precursors are chosen from a list of mono- or di- alkyl Krebs cycle intermediate precursors such as, mono-alkyl-maleates, di-alkyl-maleates, monoalkyl-citrates, or di-alkyl-citrates. Preferred Krebs cycle intermediate precursor combinations use diethylhexyl maleate and diauryl citrate. In general the amount of Krebs cycle intermediate precursor, such as diethylhexyl maleate and diauryl citrate, can be in the range of 0.5% to 10% by weight.

In preferred embodiments of the serum, an emollient that may double as a fuel substrate is used. Emollients are chosen from a list of esters, glycerol compounds, and fatty acids. Preferred embodiments include a combination of sorbitan laurate, polyglyceryl-4 laurate, PEG-60 hydrogenated oil, or olive oil.

Levels of such emollients may range from 0.05% to 50% by weight, preferably between 0.5% to 5%.

In a preferred embodiment, an antioxidant is used. Preferred antioxidants include resveratrol, antioxidants derived from green tea, and antioxidants derived from chocolate. The amount of the antioxidants, such as resveratrol, in the inventive composition can be in the range of 0.00002 to 20% by weight, preferably in the range of 0.1% to 5%.

In a preferred embodiment, vitamins are added to the serum. The preferred include niacinamide (Vitamin B3) and tetrahexyldecyl ascorbate (Vitamin C). The total amount of vitamin in the inventive combination, such as niacinamide or Vitamin C can be in the range of 0.1 to 10% by weight, preferably between 0.5 to 5%.

Vitamin B3 is advantageous because of its involvement with metabolism and role in enhancing mitochondrial function.

Vitamin C was selected for use in this system because of its multifunctional role to help as antioxidant, preservative, and rejuvenating agent for the skin.

Another category of functional ingredients are thickeners, which include polyacrylate materials. Thickeners can be present in an amount from 0.1% to 20% by weight, preferably from about 0.5% to 10% by weight.

In preferred embodiment, the serum includes a water content from 40% to 95% by weight, and preferably from 80% to 90%.

In preferred embodiment, aloe leaf powder is used. Aloe leaf powder has known skin soothing effects and promotes healing. Aloe leaf powder may be present in an amount anywhere from 0.05 to 5% by weight, preferably between 0.1% to 1%.

In preferred embodiment, amino acids are used. Amino acids can penetrate the mitochondrial membrane and then be broken down into Krebs cycle intermediates. Amino acids may be present in an amount anywhere from 0.01% to 1% by weight, preferably between 0.1 to 0.5%.

Preservatives are often added to cosmetics such as those described herein. Preservatives help to stabilize the cosmetic and prevent bacterial or fungal growth, bacterial and fungal presence and proliferation can lead to skin irritation and spoilage. Preservatives may be present in an amount anywhere from 0.05 to 5% by weight, preferably between 0.1 to 1%.

Example: Skin Serum

The following listing of ingredients are provided as an exemplary, but non-limiting, embodiment of the present disclosure, which each ingredient is then produced under special conditions to reduce contamination and preserve the active antioxidant ingredients.

Ingredients:

| Ingredient | w/w % |
|---|---|
| Distilled Water | 86.03 |
| Resveratrol Powder | 0.05 |
| Ethyl Alcohol | 1 |
| Aloe Leaf Powder | 0.4 |
| Niacinamide | 1 |
| Polysorbate 20 | 1 |
| Sorbitan Laurate, Polyglyceryl-4 Laurate, Dilauryl Citrate | 0.65 |
| PEG-60 Hydrogenated Castor Oil | 0.8 |
| Sodium Acrylate/Sodium Acryloyldimethyl Taurate | 0.65 |
| Copolymer, Isohexadecane, Polysorbate 80 | |
| Sodium Benzoate | 0.22 |
| Polysorbate 80 | 0.25 |
| Sodium Hyaluronate | 0.7 |
| Sodium Hyaluronate SLMW | 1.5 |
| Tetrahexyldecyl Ascorbate (Vitamin C) | 0.25 |
| Thiotaine | 0.25 |
| Hydrogenated Ethylhexyl Olivate and Hydrogenated Olive | 0.2 |
| Oil Unsaponifiables | |
| Diethylhexyl Maleate | 4.5 |
| Phenoxylethanol | 0.55 |
| | 100 |

A 4 week single-center, single cell, non-randomized clinical study evaluating twice-a-day application of our serum tested on 31 test subjects with a variety of skin types resulted statistically significant improvement in the areas of “Looking Younger”, “Noticeable Anti-aging Effect”, “Younger Eye Area”, “Younger Mouth Area”, “Smoother Forehead”, “Improved Confidence”, “Noticeably Decrease Wrinkles”, and “Appearance”. Furthermore, the serum has a reportedly pleasing texture and prolonged, repeated application did not change how test subjects felt about the appealing nature of the serum.

The 4-week trial reported statistically significant improvement in skin redness and even skin tone starting in day 5 persisting through the 4-week study. Skin radiance and global fine lines in facial skin area (fine wrinkles) saw statistically significant improvement by week 2 which was maintained throughout the 4-week study. Pore appearance, mottled hyperpigmentation, global wrinkles, and pinch recoil saw statistically significant improvement by the end of the 4-week study.

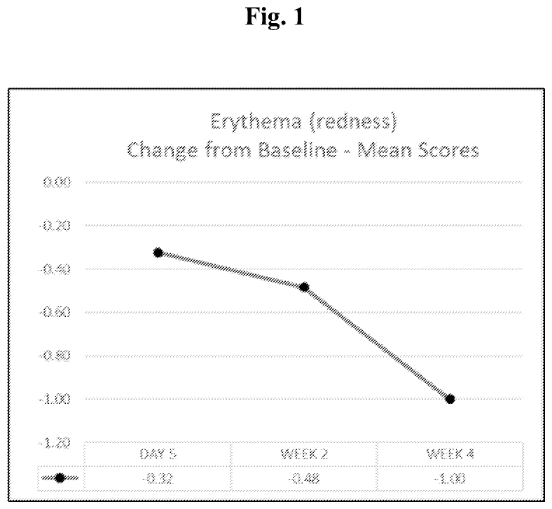

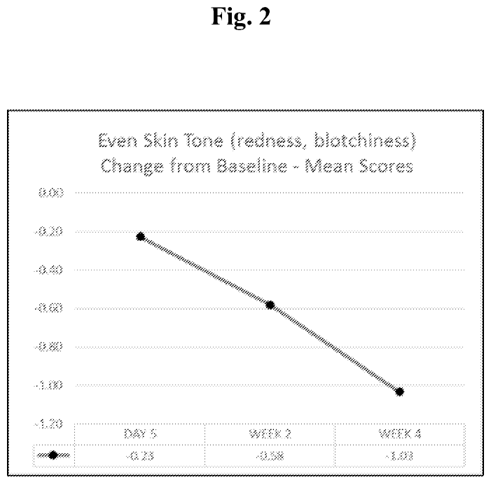

Only one test subject experienced skin irritation. Exemplary results of the 4-week study are shown in FIGS. 1-2. Table 2.0 (shown below) represents the results for FIG. 1 organized into a table.

TABLE 2.0
Full Analysis Set for Clinical Parameter
Erythema (redness)
(scale: 0 = best-9 = worst)

| NEUR Serum - 0101 | Baseline | Day 5 | Week 2 | Week 4 |
|---|---|---|---|---|
| N | 31 | 31 | 31 | 30 |
| Mean | 4.06 | 3.74 | 3.58 | 3.20 |
| Standard Deviation | 2.48 | 2.32 | 2.11 | 1.94 |
| p-value | | 0.0024 | 0.0088 | 0.0001 |
| Change from Baseline | | | | |
| Mean | | −0.32 | −0.48 | −1.00 |
| Standard Deviation | | 0.54 | 0.96 | 1.23 |
| # Improved | | 9 | 11 | 19 |
| % Improved | | 29.03% | 35.48% | 63.33% |
| # Same | | 22 | 18 | 9 |
| % Same | | 70.97% | 58.06% | 30.00% |
| # Worsened | | 0 | 2 | 2 |
| % Worsened | | 0.00% | 6.45% | 6.67% |
| p-value | | 0.0024 | 0.0088 | 0.0001 |

Table 2.5 (shown below) represents the results for FIG. 2 organized into a table.

TABLE 2.5
Full Analysis Set for Clinical Parameter
Even Skin Tone (redness, blotchiness)
(scale: 0 = best-9 = worst)

| NEUR Serum - 0101 | Baseline | Day 5 | Week 2 | Week 4 |
|---|---|---|---|---|
| N | 31 | 31 | 31 | 30 |
| Mean | 4.61 | 4.39 | 4.03 | 3.63 |
| Standard Deviation | 2.01 | 1.93 | 1.85 | 1.77 |
| p-value | | 0.0060 | 0.0002 | <.0001 |
| Change from Baseline | | | | |
| Mean | | −0.23 | −0.58 | −1.03 |
| Standard Deviation | | 0.43 | 0.76 | 1.03 |
| # Improved | | 7 | 14 | 20 |
| % Improved | | 22.58% | 45.16% | 66.67% |
| # Same | | 24 | 17 | 10 |
| % Same | | 77.42% | 54.84% | 33.33% |
| # Worsened | | 0 | 0 | 0 |
| % Worsened | | 0.00% | 0.00% | 0.00% |
| p-value | | 0.0060 | 0.0002 | <.0001 |

The unique combination of Krebs cycle precursors, Krebs cycle fuel, and antioxidants advantageously provides a significant clinical effect. A decrease in overall skin age was noticeable within 5 days and effects persistently improved over the following three weeks. Significant improvement to skin redness, radiance, and skin tone evenness where observed by within 5 days. Wrinkle reduction is visible after 2 weeks and improvements persisted through the 4-week clinical study. Skin elasticity also improved after 4 weeks of use.

The preferred embodiment involves applying a nickel-sized amount of the serum, easily achieved by dropper or similar device, onto the user's hands and smoothly rubbed onto the skin of the user. The relatively small amount necessary to achieve results leads to less product application and a lighter feel on the skin, especially when coupled with the aqueous nature of the preferred embodiments.

In preferred embodiments, the serum may be applied along with typical brands of moisturizer or makeup as normal once the serum has dried, providing minimal disruption to its users' typical skin care or makeup routine.

The disclosed embodiments are susceptible to various modifications and alternative forms, and specific examples thereof have been shown by way of example in the drawings and are herein described in detail. It should be understood, however, that the disclosed embodiments are not to be limited to the particular forms or methods disclosed, but to the contrary, the disclosed embodiments are to cover all modifications, equivalents, and alternatives.

What is claimed is:

1. A topical skin composition comprising at least one of a Krebs cycle intermediate and a Krebs cycle precursor, a topical vehicle comprising a mitochondrial fuel, and one or more antioxidants dissolved in water.

2. The topical skin composition of claim 1, wherein said topical skin composition is aqueous and comprises 0.5% to 10% by weight of the at least one of the Krebs cycle intermediate and the Krebs cycle precursor.

3. The topical skin composition of claim 1, wherein said topical skin composition is aqueous and the at least one of the Krebs cycle intermediate and the Krebs cycle precursor includes one of a mono-alkyl-maleate, a di-alkyl-maleate, a mono-alkyl-citrate, and a di-alkyl-citrate.

4. The topical skin composition of claim 1, wherein said topical skin composition is aqueous and the at least one of the Krebs cycle intermediate and the Krebs cycle precursor comprises at least dilauryl citrate and diethylhexyl maleate.

5. The topical skin composition of claim 1, wherein said topical skin composition is aqueous and the mitochondrial fuel is at least one of a sorbitan laurate, a polyglyceryl-4 laurate, a PEG-60 hydrogenated oil, and a hydrogenated or a nonhydrogenated olive oil.

6. The topical skin composition of claim 1, wherein said topical skin composition is aqueous and comprises 0.0002% to 20% by weight of the antioxidants.

7. The topical skin composition of claim 6, wherein the antioxidants includes one of a resveratrol, a green tea, or a chocolate.

8. The topical skin composition of claim 1, wherein said topical skin composition is aqueous and further comprises an emollient, a vitamin, a thickener, and one or more amino acids.

9. The topical skin composition of claim 8, wherein the emollient is selected from a group consisting of ethyl alcohol, isohexadecane, polysorbate 20, polysorbate 80, and phenoxylethanol.

10. The topical skin composition of claim 8, wherein the topical vehicle comprising a mitochondrial fuel is an emollient including one of sorbitan laurate, polyglyceryl-4-laurate, PEG-60 hydrogenated oil, or olive oil.

11. The topical skin composition of claim 10, wherein the emollient comprises 0.05% to 50% by weight.

12. The topical skin composition of claim 10, wherein the emollient comprises 0.5% to 5% by weight.

13. The topical skin composition of claim 9, wherein the vitamin includes one of a niacinamide (Vitamin B3) and a tetraxyldecyl ascorbate (Vitamin C).

14. The topical skin composition of claim 9, wherein the vitamin comprises 0.1% to 10% by weight.

15. The topical skin composition of claim 9, wherein the vitamin comprises 0.5% to 5% by weight.

16. The topical skin composition of claim 9, wherein the thickener is a polyacrylate material.

17. The topical skin composition of claim 9, wherein the thickener comprises 0.1% to 20% by weight.

18. The topical skin composition of claim 9, wherein the amino acid comprises 0.01% to 1% by weight.

19. The topical skin composition of claim 9, further comprising an aloe leaf powder.

20. The topical skin composition of claim 9, further comprising 0.05 to 5% by weight of a preservative.

21. The aqueous topical skin composition of claim 20, further comprising 0.5 to 5% by weight vitamins including one of niacinamide and tetragexyldecyl ascorbate in combination, 0.5% to 10% by weight thickeners including polyacrylate materials, 0.1% to 0.5% by weight amino acids, 0.1% to 0.5% by weight amino acids, 0.1% to 1% by weight aloe leaf powder, and 0.1% to 1% by weight preservatives, including sodium benzoate.

22. The topical skin composition of claim 9, further comprising 0.1 to 1% by weight of a preservative.

23. The aqueous topical skin composition of claim 1, wherein the water comprises 40% to 95% by weight.

24. An aqueous topical skin composition comprising 80% to 90% by weight water, 0.5 to 10% by weight of a combination of Krebs cycle intermediate precursors, diethylhexyl maleate and diauryl citrate, 0.5% to 5% of an emollient that doubles as a Krebs cycle fuel substrate and including one of a sorbitan laurate, a polyglyceryl-4-laurate, a PEG-60 hydrogenated oil, or an olive oil, and 0.01% to 5% antioxidants that includes a resveratrol, an antioxidants derived from green tea, or one or more antioxidants derived from chocolate.
